(19) United States
(12) Patent Application Publication
(10) Pub. No.: US 2025/0064437 A1
(43) Pub. Date: Feb. 27, 2025

(54) NEEDLE TUBE AND BIOPSY DEVICE

(71) Applicant: OLYMPUS MEDICAL SYSTEMS CORP., Tokyo (JP)

(72) Inventor: Ranwen LU, Tokyo (JP)

(21) Appl. No.: 18/930,343

(22) Filed: Oct. 29, 2024

Related U.S. Application Data
(63) Continuation of application No. PCT/JP2023/030647, filed on Aug. 25, 2023.

(51) Int. Cl. A61B 10/02; A61B 10/04

(52) U.S. Cl. CPC A61B 10/0275; A61B 10/04; A61B 2010/045

(57) ABSTRACT

A needle tube includes a tubular body. The body has a slit that extends in a longitudinal direction of the body from a distal end opening of the body toward a proximal end side, and that penetrates a side wall of the body in a radial direction. The length from a distal end of the body to a proximal end of the slit is 3 to 25 mm. The opening angle formed by two line segments connecting a center of the body and side ends on both sides of the slit in a width direction in a cross section perpendicular to a longitudinal axis of the body is 30° to 90°.

### CROSS-REFERENCE TO RELATED APPLICATIONS

This is a continuation of International Application PCT/JP2023/030647, with an international filing date of Aug. 25, 2023, which is hereby incorporated by reference herein in its entirety.

### TECHNICAL FIELD

The present disclosure relates to a needle tube and a biopsy device.

### BACKGROUND ART

In the related art, there is a known device used in a biopsy method under endosonography, such as endoscopic ultrasound-guided fine needle aspiration/biopsy (EUS-FNA/FNB) (for example, see Patent Literatures 1 and 2). The devices in Patent Literatures 1 and 2 each include a sheath that can be inserted into a treatment tool channel of an ultrasonic endoscope, and a needle tube that is inserted into the sheath so as to be movable forward and backward and that is pierced into biological tissue. The needle tube is thin and, for example, the diameter thereof is less than 1 mm. In Patent Literatures 1 and 2, a distal end portion of the needle tube is provided with a slit extending from the distal end of the needle tube toward the proximal end side, in order to improve the collection amount of the biological tissue.

### SUMMARY

An aspect of the present disclosure is a needle tube comprising a tubular body, wherein: the body has a slit that extends in a longitudinal direction of the body from a distal end opening of the body toward a proximal end side, and that penetrates a side wall of the body in a radial direction; a length from a distal end of the body to a proximal end of the slit is 3 to 25 mm; and an opening angle formed by two line segments connecting a center of the body and side ends on both sides of the slit in a width direction in a cross section perpendicular to a longitudinal axis of the body is 30° to 90°.

Another aspect of the present disclosure is a biopsy device comprising: a sheath; the abovementioned needle tube that is provided in the sheath so as to be able to protrude from a distal end of the sheath; and an operation portion that is provided on a proximal end side of the sheath, and that is for operating the sheath and the needle tube.

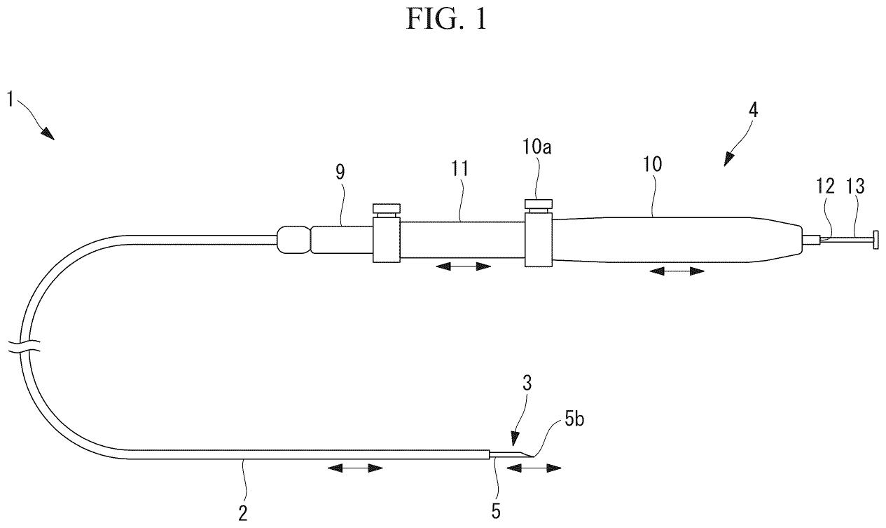

FIG. 1 is an overall configuration diagram of a biopsy device according to an embodiment of the present disclosure.

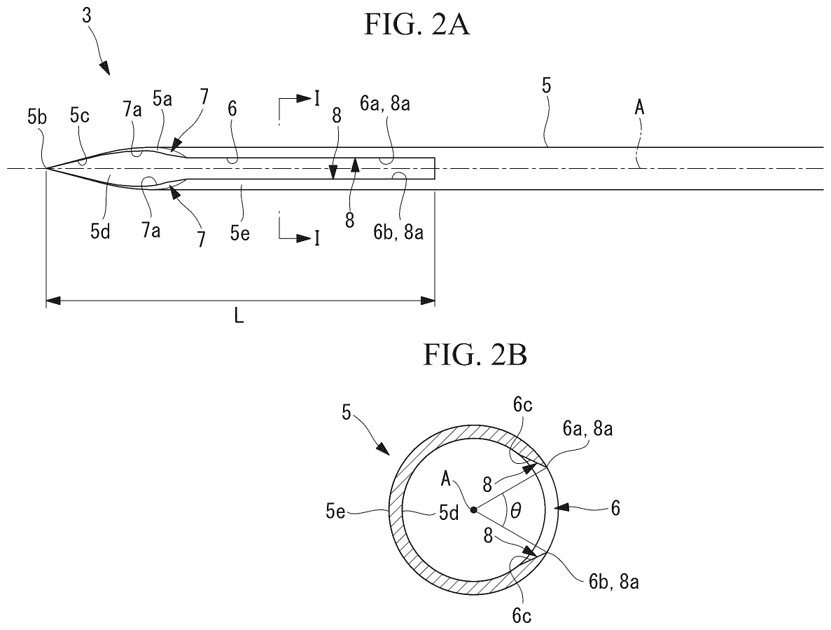

FIG. 2A is a front view of an example of a distal end-side portion of a needle tube.

FIG. 2B is a cross-sectional view, taken along the I-I line in FIG. 2A.

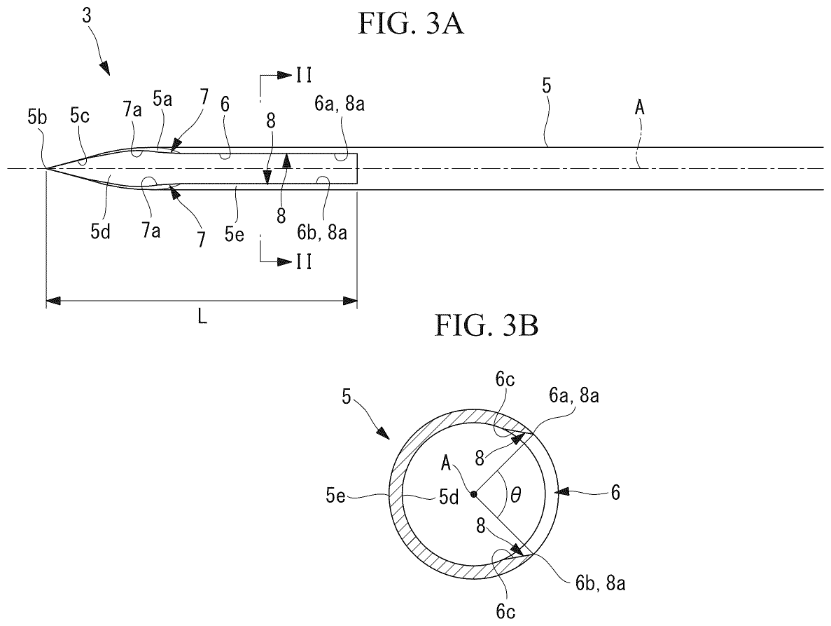

FIG. 3A is a front view of another example of the distal end-side portion of the needle tube.

FIG. 3B is a cross-sectional view, taken along the II-II line in FIG. 3A.

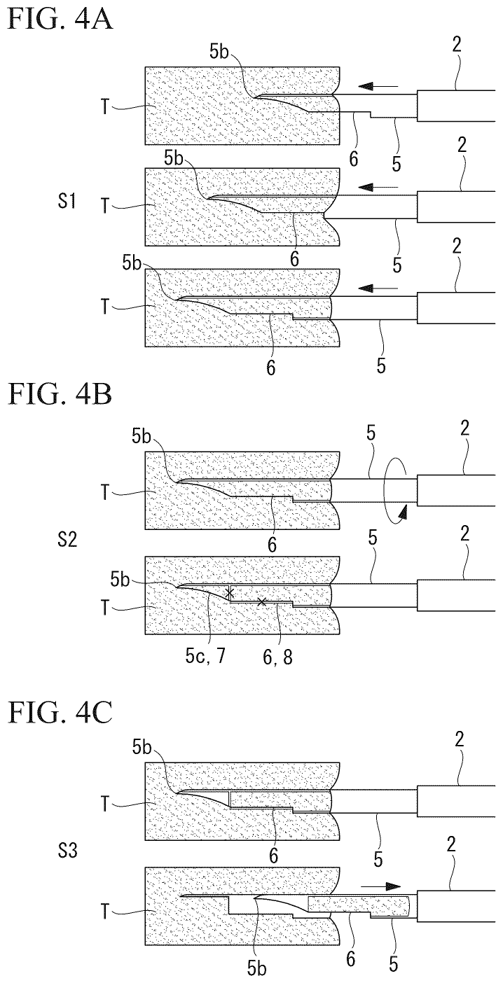

FIG. 4A is a diagram for explaining a biological tissue collection method using the biopsy device in FIG. 1, showing a step of piercing a body into biological tissue.

FIG. 4B is a diagram for explaining a biological tissue collection method using the biopsy device in FIG. 1, showing a step of rotating the body.

FIG. 4C is a diagram for explaining a biological tissue collection method using the biopsy device in FIG. 1, showing a step of removing the body from the biological tissue.

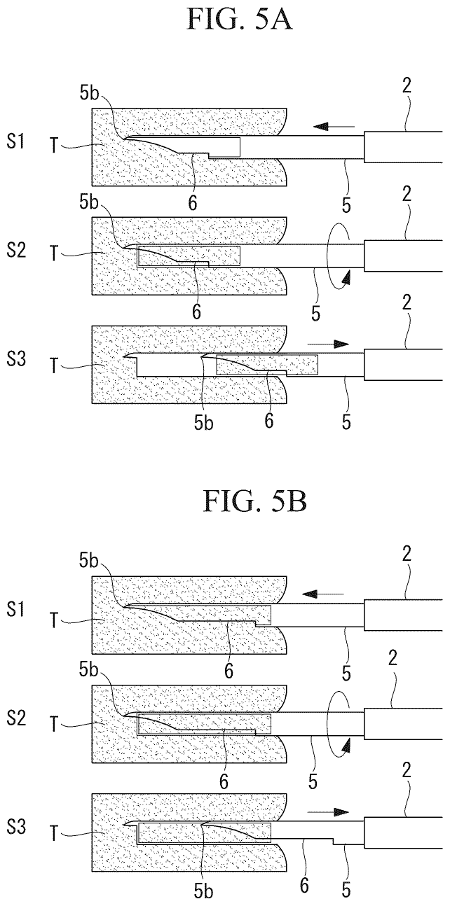

FIG. 5A is a diagram for explaining the operation of a slit having a small length L.

FIG. 5B is a diagram for explaining the operation of a slit having a large length L.

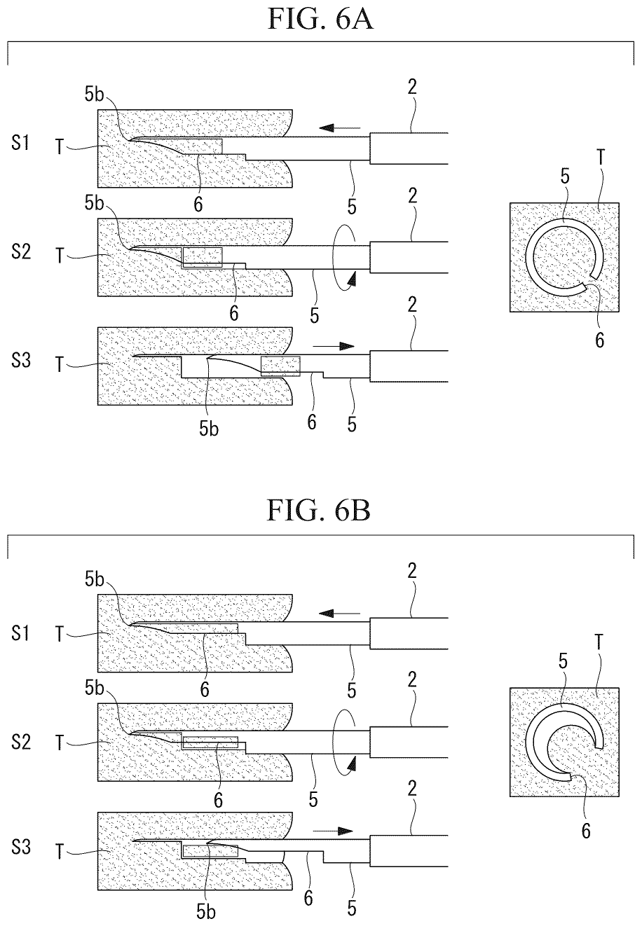

FIG. 6A is a diagram for explaining the operation of a slit having a small opening angle θ.

FIG. 6B is a diagram for explaining the operation of a slit having a large opening angle θ.

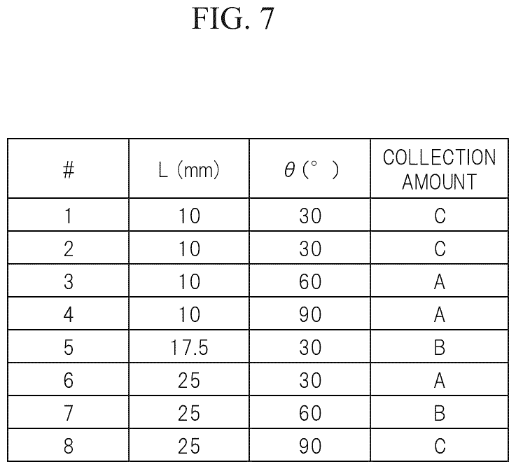

FIG. 7 is a table showing results of measuring the biological tissue collection amount using a plurality of needle tubes.

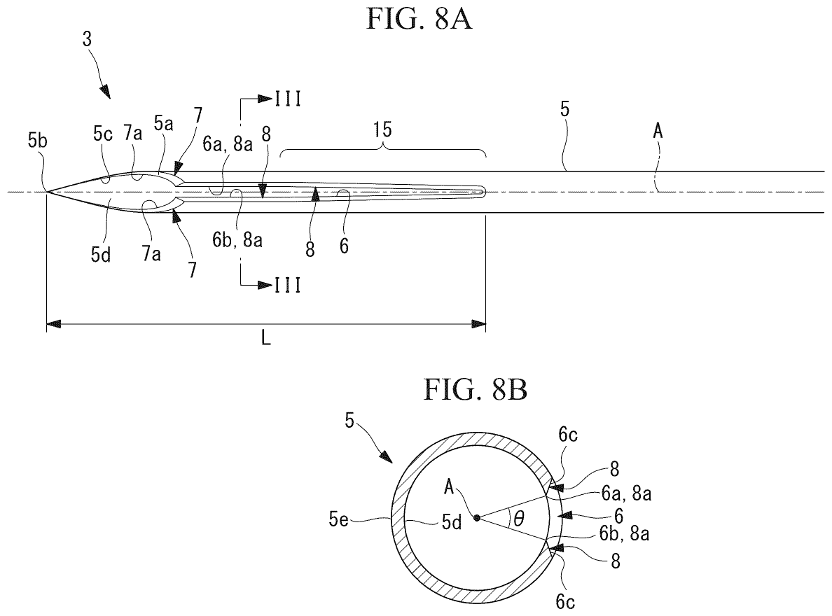

FIG. 8A is a front view of a distal end-side portion of a needle tube in which the slit has a narrow portion.

FIG. 8B is a cross-sectional view, taken along the III-III line in FIG. 8A.

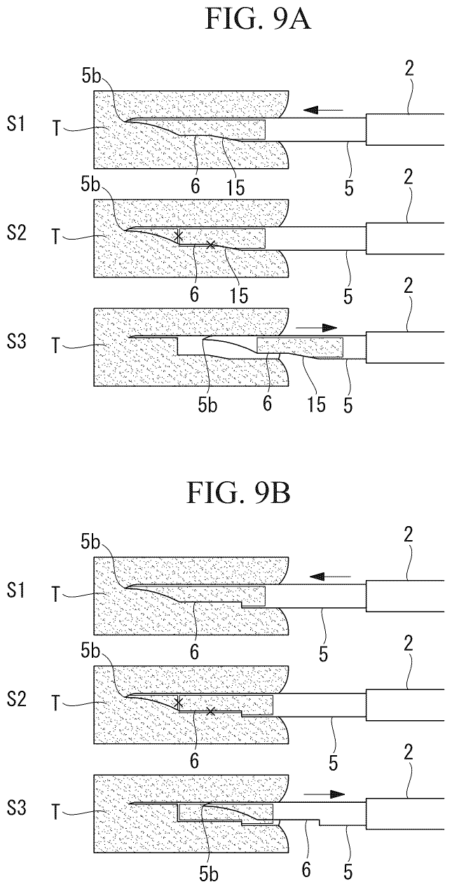

FIG. 9A is a diagram for explaining the operation of a slit that has a narrow portion.

FIG. 9B is a diagram for explaining the operation of a slit that does not have a narrow portion.

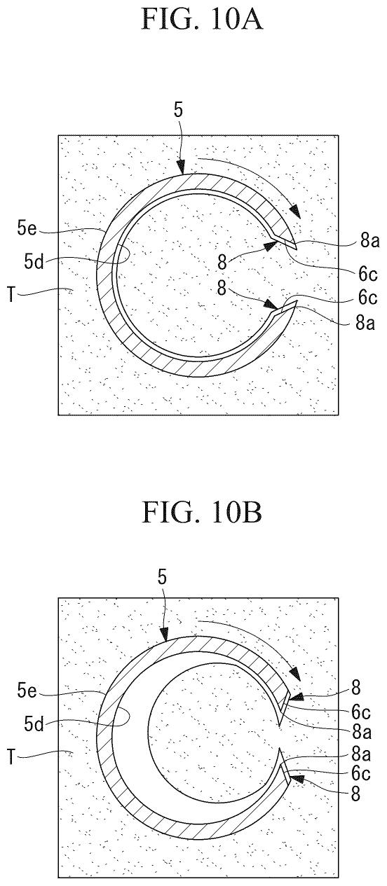

FIG. 10A is a cross-sectional view of the needle tube in which blade edges of slit blades are located at radially outer ends.

FIG. 10B is a cross-sectional view of the needle tube in which blade edges of slit blades are located at radially inner ends.

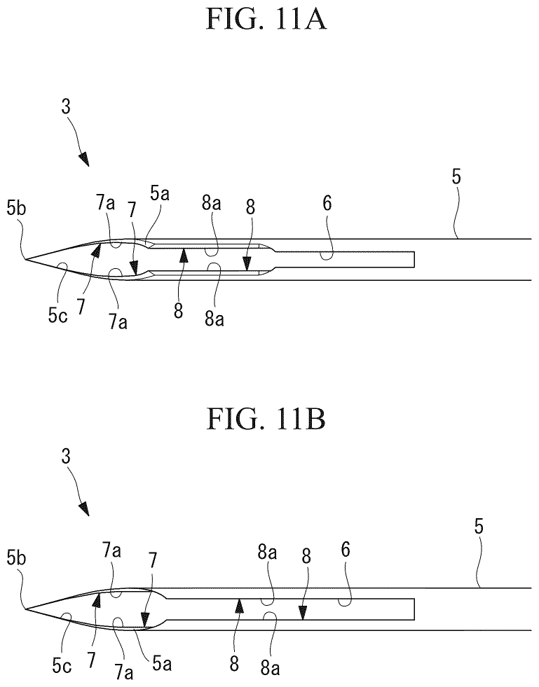

FIG. 11A is a diagram showing a modification of the slit blades.

FIG. 11B is a diagram showing a modification of distal end blades.

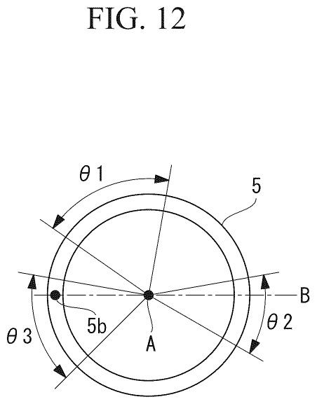

FIG. 12 is a schematic diagram for explaining a positional relationship between a needle tip and a slit when the body of the needle tube is viewed from the distal end side in a direction along a longitudinal axis.

### DESCRIPTION OF EMBODIMENT

A needle tube and a biopsy device according to an embodiment of the present disclosure will be described below with reference to the drawings.

As shown in FIG. 1, a biopsy device 1 according to this embodiment includes: a sheath 2; a biopsy needle tube 3 that is disposed in the sheath 2; and an operation portion 4 that is provided on the proximal end side of the sheath 2 and the needle tube 3.

The biopsy device 1 is used in combination with an ultrasonic endoscope. The sheath 2 and the needle tube 3 are inserted into a treatment tool channel of the ultrasonic endoscope, and the operation portion 4 is disposed outside the ultrasonic endoscope. The distal ends of the sheath 2 and the needle tube 3 protruding from the distal end of the ultrasonic endoscope are disposed within a field of view of the ultrasonic endoscope, and are observed in an optical image and an ultrasonic image acquired by the ultrasonic endoscope.

The sheath 2 is an elongated tubular member that has flexibility and that opens at both end surfaces, and has an outer diameter smaller than the inner diameter of the treatment tool channel.

The needle tube 3 has an elongated body 5 having flexibility. The body 5 is a cylindrical member that opens at both end surfaces, and has an outer diameter smaller than the inner diameter of the sheath 2. The body 5 is movable in a longitudinal direction with respect to the sheath 2, and is also rotatable about a longitudinal axis A of the body 5 with respect to the sheath 2.

FIGS. 2A and 2B show an example of the shape of a distal end-side portion of the body 5, and FIGS. 3A and 3B show another example of the shape of the distal end-side portion of the body 5. A distal end surface 5a of the body 5 is inclined relative to the longitudinal axis A, and a distal end 5b of the body 5 is a sharp needle tip.

The body 5 has a slit 6 in a distal end portion thereof. The slit 6 extends in the longitudinal direction of the body 5 from a distal end opening 5c at the distal end surface of the body 5 toward the proximal end side, and penetrates a side wall of the body 5 in a radial direction. The inner side of the body 5 is continuous with the outer side of the body 5 via the slit 6 and the distal end opening 5c. In the examples of FIGS. 2A and 3A, the slit 6 is provided at a position shifted by 180° in the circumferential direction about the longitudinal axis A with respect to the needle tip 5b; however, as will be described later, the slit 6 may be provided at another position (see FIG. 12).

Parameters for determining the shape of the body 5 include a length L and an opening angle θ. The length L is a length from the distal end 5b to the proximal end of the slit 6 in the longitudinal direction of the body 5 (see FIGS. 2A and 3A). The opening angle θ is an angle formed by two line segments connecting side ends 6a, 6b on both sides of the slit 6 in the width direction and the center (longitudinal axis A) of the body 5 in a cross section perpendicular to the longitudinal axis A (see FIGS. 2B and 3B). In the needle tube 3 of this embodiment, the length L is 3 to 25 mm and the opening angle θ is 30° to 90°.

The product of the length L and the opening angle θ is preferably within a prescribed range. In other words, as shown in FIGS. 2A and 2B and FIGS. 3A and 3B, the length L and the opening angle θ are in an inverse proportion relationship, and the opening angle θ is preferably smaller as the length L is larger. The prescribed range is set according to the dimensions such as the inner diameter, shape, etc. of the body 5.

The body 5 preferably has: distal end blades 7 provided on the distal end surface 5a of the body 5; and slit blades 8 provided on both side surfaces 6c of the slit 6, which face each other in the width direction.

Each of the distal end blades 7 is formed of an inner circumferential surface 5d of the body 5 and the distal end surface 5a of the body 5, which forms an acute angle with the inner circumferential surface 5d, and has a sharp blade edge 7a at a radially inner end of the distal end surface 5a.

As shown in FIGS. 2B and 3B, the side surfaces 6c each form an acute angle with an outer circumferential surface 5e of the body 5, and a radially outer end of the side surface 6c protrudes farther in the circumferential direction than a radially inner end of the side surface 6c. Each of the slit blades 8 is formed of the side surface 6c and the outer circumferential surface 5e, and has a sharp blade edge 8a at the radially outer end of the side surface 6c.

The side surface 6c may form an acute angle with the inner circumferential surface 5d, and the slit blade 8 may be formed of the side surface 6c and the inner circumferential surface 5d (see FIGS. 8A and 8B). In this case, the slit blade 8 has the blade edge 8a at the radially inner end of the side surface 6c.

The operation portion 4 is a portion for an operator to operate the sheath 2 and the needle tube 3, and has a body 9 and an operation member 10 provided on the body 9.

The body 9 is composed of a cylindrical member, and proximal end portions of the sheath 2 and the body 5 are housed in the body 9.

The operation member 10 is for moving the needle tube 3 with respect to the sheath 2, and is composed of a cylindrical member disposed outside the body 9. The operation member 10 is connected to the proximal end of the body 5, and is movable in the longitudinal direction with respect to the body 9 and is also rotatable about a longitudinal axis of the body 9. The operation member 10 may be fixable with respect to the sheath 2 by means of a fixing member 10a, such as a thumbscrew. The operator can advance the body 5 to the distal end side by pushing the operation member 10 to the distal end side, so that the needle tip 5b can protrude from the distal end of the sheath 2. In addition, by pulling the operation member 10 to the proximal end side, the operator can retract the body 5 to the proximal end side up to a position where the needle tip 5b is stored in the sheath 2.

The operation portion 4 may further include another operation member 11, a suction port 12, and a stylet 13. The operation member 11 is for moving the sheath 2 with respect to the body 9, is connected to the proximal end of the sheath 2, and is movable in the longitudinal direction with respect to the body 9. The suction port 12 opens at the proximal end of the body 9 and communicates with the inside of the body 5, and a suction tool, such as a syringe, can be connected to the suction port 12. The stylet 13 can be inserted into the body 5 from the suction port 12.

Next, the operation of the needle tube 3 and the biopsy device 1 will be described.

The biopsy device 1 is used in combination with an ultrasonic endoscope in endoscopic ultrasound-guided fine needle aspiration/biopsy (EUS-FNA/FNB).

As shown in FIGS. 4A to 4C, a biological tissue collection method using the biopsy device 1 includes: step S1 of piercing the body 5 into biological tissue T; step S2 of rotating the body 5; and step S3 of removing the body 5 from the biological tissue T.

Prior to step S1, an ultrasonic endoscope is inserted into a body cavity, and the ultrasonic endoscope is disposed at a position where target biological tissue T, for example, a pancreatic tumor is observed. Next, the sheath 2 storing the needle tube 3 is inserted into a treatment tool channel of the ultrasonic endoscope, and the distal end of the sheath 2 protruding from a distal end opening of the treatment tool channel is disposed at an appropriate position with respect to the biological tissue T.

Next, as shown in FIG. 4A, as a result of pushing the operation member 10, the body 5 protrudes from the distal end of the sheath 2 and is pierced into the biological tissue T (step S1). At this time, the biological tissue T enters deep into the body 5 due to the presence of the slit 6.

Next, as shown in FIG. 4B, as a result of rotating the operation member 10, the body 5 is rotated (step S2). The biological tissue T in the body 5 rotates together with the body 5 by friction between the biological tissue T in the body 5 and the inner circumferential surface 5d. By doing so, the biological tissue T is cut in the slit 6 and broken by torsion at the distal end opening 5c, and thus, the biological tissue T in the body 5 is separated from the biological tissue T outside the body 5. At this time, the biological tissue T is easily cut by means of the distal end blades 7 and the slit blades 8.

Next, as shown in FIG. 4C, as a result of pulling the operation member 10, the body 5 is pulled out from the biological tissue T and completely stored in the sheath 2 (step S3).

Subsequently, by pulling out the sheath 2 storing the body 5 from the treatment tool channel, the needle tube 3 is removed from inside the body. By doing so, the biological tissue T taken into the body 5 is collected.

In this case, with this embodiment, a large amount of the biological tissue T is taken into the body 5, at the time of piercing, by means of the slit 6 provided in the body 5, as compared with a case in which the slit 6 is not provided. With this configuration, it is possible to improve the collection amount of the biological tissue T.

Furthermore, with this embodiment, as a result of setting the length L to 3 to 25 mm and the opening angle θ to 30° to 90°, it is possible to further improve the collection amount of the biological tissue T, and to reliably collect a sufficient amount of the biological tissue T.

FIGS. 5A and 5C are for explaining a difference in the collection amount of the biological tissue T due to a difference in the length L, and FIG. 5A shows a case where the length L is small and FIG. 5B shows a case where the length L is large.

At the time of piercing, as the length L is larger, the biological tissue T enters deeper into the body 5 and a larger amount of the biological tissue T is taken into the body 5. Meanwhile, as the length L is larger, a contact area between the biological tissue T in the body 5 and the biological tissue T outside the body 5, that is, the friction increases. Thus, as the length L is larger, it becomes easier for the biological tissue T to slip off from inside the body 5 through the distal end opening 5c during removal of the body 5.

In the case in which the length L is 3 to 25 mm, taking a sufficient amount of the biological tissue T into the body 5 at the time of piercing and suppressing the slip-off of the biological tissue T during the removal can be both achieved. With this configuration, it is possible to obtain a sufficient collection amount even when the biological tissue T contains a large amount of moisture and is slippery with respect to the body 5.

In a case in which the length L is smaller than 3 mm or larger than 25 mm, it is difficult to take a sufficient amount of the biological tissue T into the body 5 at the time of piercing, or the biological tissue T slips off from inside the body 5 during the removal, thus making it difficult to obtain a sufficient collection amount.

FIGS. 6A and 6B are for explaining a difference in the collection amount of the biological tissue T due to a difference in the opening angle θ. FIG. 6A shows a case where the opening angle θ is small and FIG. 6B shows a case where the opening angle θ is large.

At the time of piercing, as the opening angle θ is larger, the biological tissue T can easily enter the body 5 and a larger amount of the biological tissue T is taken into the body 5. Meanwhile, as the opening angle θ is larger, it becomes easier for the biological tissue T to slip off from inside the body 5 through the slit 6 during rotation of the body 5 (see a right diagram for step S2). In addition, as the opening angle θ is larger, the contact area between the biological tissue T in the body 5 and the biological tissue T outside the body 5, that is, the friction increases. Thus, as the opening angle θ is larger, it becomes easier for the biological tissue T to slip off from inside the body 5 through the distal end opening 5c during the removal.

In the case in which the opening angle θ is 30° to 90°, taking a sufficient amount of the biological tissue T into the body 5 at the time of piercing and suppressing the slip-off of the biological tissue T during the rotation and removal can be both achieved. With this configuration, it is possible to obtain a sufficient collection amount even when the biological tissue T contains a large amount of moisture and is slippery with respect to the body 5.

In a case in which the opening angle θ is smaller than 30° or larger than 90°, it is difficult to take a sufficient amount of the biological tissue T into the body 5 at the time of piercing, or the biological tissue T slips off during the rotation and removal, thus making it difficult to obtain a sufficient collection amount.

FIG. 7 shows results of experiments in which the collection amount of the biological tissue T was measured using needle tubes 3 (#1 to #8) having various lengths L and opening angles θ. The thickness of the body 5 used in the experiments was 19 G (inner diameter of approximately 0.7 mm), and the biological tissue T was collected through the piercing, rotation, and removal of the body 5. The collection amount was evaluated in three levels, namely, “A (excellent)”, “B (good)”, and “C (fair)”, according to the weight of the collected biological tissue T. “A” indicates that the collection amount is the largest, and “C” indicates that the collection amount is the smallest.

As can be seen from FIG. 7, the biological tissue T was successfully collected by all the needle tubes #1 to #8. The collection amount was large in the needle tubes in which length L×opening angle θ is greater than 300 and equal to or less than 1500, and the collection amount was particularly large in the needle tubes in which length L×opening angle θ is within the range of 600 to 900. Therefore, length L×opening angle θ is preferably greater than 300 and equal to or less than 1500, and is more preferably within the range of 600 to 900.

In this embodiment, the slit 6 preferably has a narrow portion 15 having a width smaller than that of the other portion of the slit 6. The narrow portion 15 is provided in a proximal end portion of the slit 6. For example, as shown in FIGS. 8A and 8B, the width of the slit 6 may gradually decrease, in a continuous manner, from an intermediate position in the longitudinal direction toward the proximal end, whereby the narrow portion 15 may be formed in the proximal end portion of the slit 6. The width may decrease toward the proximal end in a stepwise manner, or may gradually decrease from the distal end of the slit 6 to the proximal end thereof.

FIGS. 9A and 9B are for explaining a difference in the collection amount of the biological tissue T due to the presence or absence of the narrow portion 15. FIG. 9A shows a case where the narrow portion 15 is provided and FIG. 9B shows a case where the narrow portion 15 is not provided.

As the width of the slit 6 is smaller, a contact area between the biological tissue T in the body 5 and the inner circumferential surface 5d, that is, the friction increases. Therefore, a holding force of the body 5 with respect to the biological tissue T in the body 5 increases in the narrow portion 15, and an anchor effect of more firmly holding the biological tissue T in the body 5 is exhibited. Furthermore, the contact area between the biological tissue T in the body 5 and the biological tissue T outside the body 5 is reduced due to the presence of the narrow portion 15, whereby the biological tissue T is prevented from slipping off from inside the body 5 during the removal. With this configuration, it is possible to more reliably prevent the biological tissue T from slipping off from inside the body 5 during the removal, and to further improve the collection amount of the biological tissue T.

In this embodiment, the blade edges 8a of the slit blades 8 are preferably located at the radially outer ends of the side surfaces 6c. FIGS. 10A and 10B show a cross section of the body 5, which is perpendicular to the longitudinal axis A. In FIG. 10A, the blade edges 8a are located at the radially outer ends of the side surfaces 6c, that is, on the outer circumferential surface 5e. In FIG. 10B, the blade edges 8a are located at the radially inner ends of the side surfaces 6c, that is, on the inner circumferential surface 5d.

In the case in which the blade edges 8a are located at the radially outer ends, the biological tissue T in the body 5 is less likely to slip off through the slit 6 during the rotation of the body 5 (see FIG. 10A). Meanwhile, in the case in which the blade edges 8a are located at the radially inner ends, the biological tissue T in the body 5 easily slips off through the slit 6 during the rotation of the body 5 (see FIG. 10B).

Therefore, as a result of the blade edges 8a being located at the radially outer ends, it is possible to further improve the collection amount of the biological tissue T.

In this embodiment, as shown in FIG. 11A, the position of each of the blade edges 8a may change from the radially outer end of the side surface 6c to the radially inner end thereof at an intermediate position in the longitudinal direction of the slit blade 8. In FIG. 11A, the blade edge 8a is located at the radially outer end on the proximal end side and is located at the radially inner end on the distal end side.

Similarly, as shown in FIG. 11B, the position of each of the blade edges 7a may change from the radially inner end of the distal end surface 5a to the radially outer end thereof at an intermediate position in the longitudinal direction of the distal end blade 7. In FIG. 11B, the blade edge 7a is located at the radially outer end on the proximal end side and is located at the radially inner end on the distal end side.

In this embodiment, the slit 6 is disposed at a position shifted by 180° with respect to the needle tip 5b; however, the position of the slit 6 is not limited thereto, and the slit 6 may be disposed at another position.

Specifically, the slit 6 can be provided in an arbitrary range that does not overlap the position of the needle tip 5b, when viewed from the distal end side in a direction along the longitudinal axis A. The abovementioned effect can be obtained as long as the slit 6 is provided in such a range.

FIG. 12 schematically shows examples of the positional relationship between the needle tip 5b and the slit 6, when viewed from the distal end side in the direction along the longitudinal axis A. For example, the slit 6 may be provided in a range indicated by θ1 or θ2. The slit 6 is preferably provided at a position where a straight line B passing through the needle tip 5b and the longitudinal axis A passes through the slit 6 (for example, a position indicated by θ2).

The range indicated by θ3, which includes the needle tip 5b, is inappropriate to provide the slit 6.

Although the embodiment of the present disclosure and the modifications thereof have been described above with reference to the drawings, the specific configuration of the present disclosure is not limited to the abovementioned embodiment and modifications, and various design changes can be made within a range that does not depart from the scope of the present disclosure. In addition, the components illustrated in the abovementioned embodiment and modifications can be combined as appropriate.

For example, the needle tube and the biopsy device according to the present disclosure may be applied to any biopsy method other than the EUS-FNA/FNB, and rigid members may be employed.

The present disclosure affords an advantage in that it is possible to improve a collection amount of biological tissue.

### REFERENCE SIGNS LIST

1 biopsy device
2 sheath
3 needle tube
5 body
5a distal end surface
5b distal end
5c distal end opening
5d inner circumferential surface
5e outer circumferential surface
6 slit
6a, 6b side end
6c side surface
7 distal end blade
7a blade edge
8 slit blade
8a blade edge
15 narrow portion
A longitudinal axis
L length
θ opening angle
T biological tissue

1. A needle tube comprising a tubular body, wherein:
the body has a slit that extends in a longitudinal direction of the body from a distal end opening of the body toward a proximal end side, and that penetrates a side wall of the body in a radial direction;
a length from a distal end of the body to a proximal end of the slit is 3 to 25 mm; and
an opening angle formed by two line segments connecting a center of the body and side ends on both sides of the slit in a width direction in a cross section perpendicular to a longitudinal axis of the body is 30° to 90°.

2. The needle tube according to claim 1, wherein the slit has a narrow portion in a proximal end portion thereof, and the narrow portion has a width smaller than that of another portion of the slit.

3. The needle tube according to claim 1, wherein:
the body has a slit blade that is formed of an outer circumferential surface of the body and a side surface of the slit, which forms an acute angle with the outer circumferential surface; and
a blade edge of the slit blade is located at a radially outer end of the side surface.

4. The needle tube according to claim 1, wherein a product of the length and the opening angle is within a prescribed range.

5. The needle tube according to claim 1, wherein:
the body has a slit blade that is formed of an outer circumferential surface or an inner circumferential surface of the body and a side surface of the slit, which forms an acute angle with the outer circumferential surface or the inner circumferential surface; and
a position of the blade edge of the slit blade changes from a radially outer end of the side surface to a radially inner end thereof at an intermediate position in the longitudinal direction.

6. The needle tube according to claim 1, wherein:
the body has a distal end blade that is formed of an outer circumferential surface or an inner circumferential surface of the body and a distal end surface of the body, which forms an acute angle with the outer circumferential surface or the inner circumferential surface; and
a position of a blade edge of the distal end blade changes from a radially outer end of the distal end surface to a radially inner end thereof at an intermediate position in the longitudinal direction.

7. The needle tube according to claim 1, wherein:
the body has a sharp needle tip at the distal end thereof; and
the slit is provided at a position that does not overlap the needle tip, when the body is viewed in a direction along the longitudinal axis.

8. The needle tube according to claim 7, wherein the slit is provided at a position where a straight line passing through the needle tip and the longitudinal axis passes through the slit, when the body is viewed in the direction along the longitudinal axis.

9. A biopsy device comprising:
a sheath;
the needle tube according to claim 1, which is provided in the sheath so as to be able to protrude from a distal end of the sheath; and
an operation portion that is provided on a proximal end side of the sheath, and that is for operating the sheath and the needle tube.
